(19) United States
(12) Patent Application Publication
(10) Pub. No.: US 2025/0388727 A1
(43) Pub. Date: Dec. 25, 2025

(54) MIXING VESSEL FOR HYDROLYSIS AND FERMENTATION OF CELLULOSE AND PROCESS USING SUCH

(71) Applicant: SixRing Inc., Calgary (CA)

(72) Inventors: Markus Weissenberger, Calgary (CA); Tenzin Leksmon, Calgary (CA); Alejandra Enriquez, Calgary (CA); Julie Greer, Calgary (CA)

(21) Appl. No.: 19/176,412

(22) Filed: Apr. 11, 2025

(30) Foreign Application Priority Data
Jun. 21, 2024 (CA) 3244167

(51) Int. Cl. C08H 8/00; C12N 9/42; C12P 19/02

(52) U.S. Cl. CPC C08H 8/00; C12N 9/2437; C12P 19/02

(57) ABSTRACT

A process for the liquefaction of cellulose, said process comprising:
providing a source of cellulose; wherein said source of cellulose comprising cellulose and hemicellulose, wherein said source of cellulose is non-flowable;
adding said source of cellulose to a mixing vessel; wherein said mixing vessel comprises a mixing apparatus selected from the group consisting of: a horizontal single helicoid auger flight; a horizontal double helicoid auger flight; a vertical single helicoid auger flight; a vertical double helicoid auger flight; a horizontal single ribbon auger flight; a horizontal double ribbon auger flight; a vertical single ribbon auger flight; a vertical double ribbon auger flight; a horizontal single sectional auger flight: a horizontal double sectional auger flight; and a vertical single sectional auger flight; a vertical double sectional auger flight;
adding a solution to said mixing vessel containing said source of cellulose to yield a reaction mixture wherein said source of cellulose is present in a concentration of solids ranging from 5% to 50% of the total mass of said reaction mixture;
mixing said reaction mixture in said mixing vessel until said reaction mixture has undergone liquefaction to yield a resulting mixture comprising oligomers and monomers from the degradation of cellulose and hemicellulose and wherein said resulting mixture has a resulting viscosity of no more than 15 Pa·s; and
optionally, separating said resulting mixture into a liquid stream used for subsequent processing and a solid stream that is introduced back into the mixing vessel for further processing.

### FIELD OF THE INVENTION

The present invention is directed to the processing of a high solids content biomaterial into a liquid, more specifically, there is provided a process to liquefy a high solids content source of cellulose into a low viscosity partially saccharified mixture.

### BACKGROUND OF THE INVENTION

Biofuel is increasingly becoming a necessity in order to wean off the human consumption of fossil fuels in aspects of everyday life, transport and home heating being the largest two industries of focus. As an alternative energy source to oil and coal, the main feedstock for bioethanol production is starch, which can yield its sugar much more readily than cellulose. This is due to the difference in structure as starch links glucose molecules together through alpha-1,4 linkages and cellulose links glucose with beta-1,4 linkages. The beta-1,4 linkages allow for crystallization of the cellulose, leading to a more rigid structure which is more difficult to break down.

The limitation that comes from solely using the sugars from starches for the production of biofuels such as bioethanol prevents the utilization of the larger portion of biomass, which comes in the form of lignocellulosic biomass (contains lignin, cellulose and hemicellulose) present in almost every plant on earth. A delignification reaction allows the recovery of the carbohydrate-based portion (cellulose and hemicellulose) from those lignocellulosic plants. Once the cellulose is separated from the other two biomass constituents i.e., lignin, and hemicellulose, further degradation of the cellulose generates cellobiose and/or glucose, which can be further processed to bioethanol.

Bioethanol is seen a sustainable alternative that can be used directly or added to gasoline to reduce GHG emissions. With the goal of alleviating many countries' dependence on foreign oil, the bioethanol industry is still hampered by its dependence on corn or sugar cane as their main sources of biomass. It is estimated that about 45% of all corn production in the U.S. is directed to bioethanol fuel production. This is a situation which has disastrous consequences when the prices of gasoline goes so low as to make corn-based biofuel unsustainable on a price viewpoint.

To pivot from starches to cellulose for the production of glucose is preferable as it will provide near-unlimited amount of feedstock from waste biomass and reduce the competition with food to generate glucose. However, the costs to do so are currently prohibitive. Cellulosic ethanol, as it is called, relies on the non-food part of a plant to be used to generate ethanol. This would allow the replacement of the current more widespread approach of making bioethanol by using corn or sugarcane.

The diversity and abundance of these types of cellulose-rich plants would allow to maintain food resources mostly intact and capitalize on the waste generated from these food resources (such as cornstalk) to generate ethanol. Other cellulose sources such as grasses, algae and even trees fall under the cellulose-rich biomass, which can be used in generating ethanol if a commercially viable process is developed.

The hydrolysis of cellulose is, as seen from the above, limited by the structure of cellulose itself but also by the approaches taken to degrade to glucose. The production of a robust, low-energy, low-cost process from lignocellulosic biomass and/or cellulose has not yet been achieved.

The benefits of bioethanol are estimated to have the potential to reduce gas emissions by up to 85% over reformulated gasoline. However, numerous production challenges to generate bioethanol from lignocellulosic biomass rather than from starch have led experts in the field to conclude that, in the near future, cellulosic ethanol will not be produced in sufficient quantities to provide at least a partial gasoline replacement or alternative. It is important for bioethanol production to pivot towards use of lignocellulosic biomass as a starting material (what is known as second-generation or 2G ethanol) in order to render it environmentally desirable and economically feasible.

Lignocellulosic biomass is a widely available resource which can be used in bioethanol production.

European patent application no. 2580245A1 discloses a process of fractionation of biomass to obtain lignin, cellulose and hemicelluloses, the process comprises: a. contacting the biomass with 5% to 30% (v/v) aqueous ammonia at a temperature ranging from 50° C. to 200° C. to obtain a first biomass slurry; b. filtering the first biomass slurry to obtain a first filtrate comprising lignin and a first residue comprising cellulose and hemicellulose; c. contacting the first residue with 30% to 90% (v/v) aqueous ammonia at a temperature ranging from 50° C. to 200° C. time to obtain a second biomass slurry; and d. filtering the second biomass slurry to obtain a second filtrate comprising hemicelluloses and a second residue comprising cellulose.

U.S. patent application No. 20210348202A1 discloses a method of processing lignocellulosic biomass comprising: providing soft lignocellulosic biomass feedstock; pretreating the feedstock at pH within the range 3.5 to 9.0 in a single-stage pressurized hydrothermal pretreatment to low severity such that the pretreated biomass is characterized by having a xylan number of 10% or higher; separating the pretreated biomass into a solid fraction and a liquid fraction; hydrolysing the solid fraction with or without addition of supplemental water content using enzymatic hydrolysis catalysed by an enzyme mixture comprising endoglucanase, exoglucanase, B-glucosidase, endoxylanase, xylosidase and acetyl xylan esterase activities; and subsequently mixing the separated liquid fraction, and the hydrolysed solid fraction, whereby xylo-oligomers in the liquid fraction are degraded to xylose monomers by the action of enzyme activities remaining within the hydrolysed solid fraction.

U.S. patent application No. 20100317070A1 discloses a process for converting lignocellulosic materials which are field residues such as cotton stalks and corn stover, process residues such as sugarcane bagasse and sweet sorghum bagasse, woody parts of energy crops such as switchgrass and miscanthus, forest residues or by-products of the wood processing industries such as sawdust from sawmills to a liquid biofuel by a series of processing steps wherein the feed materials are hydrolysed in three stages and withdrawn as three product streams each consisting of solubilized fragments of one of the three major components of the feed materials and a set of concurrently operating processing steps wherein each of the three product streams is transformed through chemical or biochemical processes into products, such as pure lignin and ethanol, that have a high calorific value and process wherein these products with high calorific value are combined to form a liquid biofuel.

U.S. Pat. No. 5,336,819A teaches a process for converting cellulose to hydrocarbon product comprising subjecting the cellulose to a temperature of from 320° to 380° C. and a pressure of at least 40 atmospheres in the presence of a nickel catalyst and a cellulose derived oil without the use of any additional reducing species to produce said hydrocarbon product; said cellulose derived oil being obtained from said hydrocarbon product.

U.S. Pat. No. 9,365,778B2 teaches a process for liquefying a cellulosic material to produce a liquefied product, said process comprising: contacting the cellulosic material simultaneously with (a) an acid catalyst in an amount in a range of 4 wt % to 40 wt % based on the weight of cellulosic material; (b) a solvent mixture containing water and a co-solvent, wherein the co-solvent comprises one or more polar solvents and in an amount of equal to or more than 10% by weight and less than or equal to 95% by weight, based on the total weight of water and co-solvent; (c) a hydrogenation catalyst; and (d) a source of hydrogen; to produce a liquefied product wherein the liquefied product comprises monomeric compounds selected from the group consisting of tetrahydropyran, substituted furane compounds, substituted tetrahydrofurane compounds, substituted tetrahydropyran compounds, substituted phenol compounds, substituted guaiacol compounds, substituted syringol compounds, and any combination thereof.

US patent application US20080072478A1 discloses a process for producing a fuel product from biomass, comprising the following steps: (a) providing a biomass feedstock to a high-shear mixer; (b) mixing said biomass feedstock in said mixer in the absence of oxygen and under conditions sufficient for the biomass to undergo liquefaction, thereby forming liquefied biomass; and (c) re-circulating and blending at least a portion of said liquefied biomass with said feedstock biomass, wherein said mixing said organic-waste material in the absence of oxygen in said extruder is carried out at a temperature of at least 650 degrees F. and at a pressure of at least 200 psi.

U.S. Pat. No. 9,127,402B2 teaches a method for liquefying biomass, comprising: (a) mixing a solid organic ammonium salt containing single nitrogen with at least one organic compound which is capable of forming a hydrogen bond with the solid organic ammonium salt to form a first mixture; (b) heating the first mixture until the first mixture becomes a solution; (c) mixing a biomass and an acid catalyst with the solution to form a second mixture; and (d) heating the second mixture to make the biomass therein convert into a liquefied product.

U.S. Pat. No. 10,533,503 B2 discloses a system for treating biomass for the production of ethanol and a biorefinery for producing a fermentation product from biomass. The biorefinery comprises a system for preparing the biomass into prepared biomass and system for pre-treating the biomass into pre-treated biomass. The biorefinery comprises a separator, a first treatment system, a second treatment system, and a fermentation system. A method for producing a fermentation product from biomass is also disclosed.

U.S. Pat. No. 9,902,982 B2 provides a continuous process for enzymatic hydrolysis of pretreated biomass, the process comprising: providing a pretreated lignocellulosic biomass feed material containing cellulose; introducing the pretreated lignocellulosic biomass feed material to a mechanical-treatment unit containing one or more decompression regions configure to release pressure; introducing a liquid solution containing cellulase enzymes to one or more decompression regions in the mechanical-treatment unit, wherein the liquid solution enters void spaces between fibers of the pretreated lignocellulosic biomass feed material, to form enzyme-containing cellulose-rich solids; and retaining the enzyme-containing cellulosic rich solids under effective hydrolysis conditions to hydrolyze at least some of the cellulose to glucose. Various apparatus configurations are disclosed for the mechanical-treatment unit.

It is known to those skilled in the art that a high cellulosic solids content is optimal in saccharification and subsequent or simultaneous fermentation processes to maximize the titer of the value-added chemical in the solution mixture and minimize costs associated with its production. When said value-added chemical is ethanol, it is known that common yeast such as Saccharomyces Cerevisiae can only tolerate ethanol titers of up to 10-15% vol. depending on the strain, thus having a low cellulosic loading (<10%) will not maximize the ability of the yeast to produce alcohol considering the conversion of cellulose to ethanol. Furthermore, the lower the titer of the value-added chemical in the final stream, the higher the costs associated with its purification (i.e., distillation and dehydration costs). To minimize these costs and maximize production, it is desirable to increase the cellulose loading to 15 to 30% wt. or higher when possible.

One of the primary limitations of large-scale cellulosic ethanol production is the amount of cellulose that can be processed at a given time. This is because at high solids loadings (>5-10%), the viscosity of the mixture makes it difficult to process using traditional equipment. Cellulose fibers are hydrophilic by nature and will bond with water via hydrogen bonding. This causes cellulose to be extremely absorbent and at higher loadings (>10%), will begin to form a thick slurry that is difficult to mix properly using traditional equipment. At loadings of >15% wt., the mixture displays the physical characteristics of a solid. In addition, this creates a problem for the enzymatic hydrolysis, as the enzymes will not be evenly distributed throughout the reaction mixture.

In light of the state-of-the-art with respect to the use of lignocellulosic biomass to generate products such as organic-based fuels (including but not limited to bioethanol and biofuels), there still exists a need for a process which is capable of being scaled up efficiently which results in streams of separated lignocellulosic biomass constituents which can then be used, for example, in the manufacturing of such fuels. Additionally, there still exists a need to apply said process to a high purity cellulose comprising a low lignin content, wherein the efficiency of the enzymatic liquefaction and saccharification are not in jeopardy due to the presence of inhibitors obtained from the hydrolysis of lignin and hemicellulose.

According to a preferred embodiment of the present invention, this challenge was overcome by using a horizontal cylindrical reactor equipped with a rotating auger and a variable frequency device capable of changing the motor speed and direction (forward/reverse) was utilized. The slow auger rotation allows for thicker slurries containing large amounts of cellulose to be mixed, therefore allowing for even degradation liquefaction and increased liquefaction rates. The combination of this mechanical process approach with a cellulosic feedstock having a high purity allows to generate a mixture of depolymerized cellulose which can be readily converted to various other chemicals, such as, but not limited to, ethanol.

Following liquefaction, the mixture can be pumped into a reactor to undergo simultaneous saccharification and fermentation (SSF) or separate hydrolysis and fermentation (SHF). This combination of a primary liquefaction step, followed by a secondary SSF reaction step is known as hybrid hydrolysis and fermentation (HHF).

### SUMMARY OF THE INVENTION

According to a first aspect of the present invention, there is provided a process to liquefy a high solids content reaction mixture comprising a highly pure source of cellulose as an intermediate step between the delignification of a biomass comprising cellulose and a subsequent conversion step of liquefied cellulose fragments into other chemicals.

According to a preferred embodiment of the present invention, said highly pure source of cellulose, being a cellulose having a low hemicellulose content and a low lignin content, can be used to generate a liquid mixture of depolymerized cellulose comprising cellulose oligomers, cellobiose and glucose.

According to an aspect of the present invention, there is provided a process for the liquefaction of cellulose, said process comprising:
providing a source of cellulose; wherein said source of cellulose comprises cellulose and hemicellulose, wherein said source of cellulose is non-flowable;
adding said source of cellulose to a mixing vessel; wherein said mixing vessel comprises a mixing apparatus selected from the group consisting of: a horizontal single helicoid auger flight; a horizontal double helicoid auger flight; a vertical single helicoid auger flight; a vertical double helicoid auger flight; a horizontal single ribbon auger flight; a horizontal double ribbon auger flight; a vertical single ribbon auger flight; a vertical double ribbon auger flight; a horizontal single sectional auger flight: a horizontal double sectional auger flight; and a vertical single sectional auger flight; a vertical double sectional auger flight;
adding a solution to said mixing vessel containing said source of cellulose to yield a reaction mixture wherein said source of cellulose is present in a concentration of solids ranging from 5% to 50% of the total mass of said reaction mixture; and wherein said reaction mixture is non-flowable;
mixing said reaction mixture in said mixing vessel until said reaction mixture has undergone liquefaction to yield a resulting mixture comprising oligomers and monomers from the degradation of cellulose and hemicellulose and wherein said resulting mixture has a viscosity of no more than 15 Pa's; and
optionally, separating said resulting mixture into a liquid stream used for subsequent processing and a solid stream that is introduced back into the mixing vessel for further processing.

According to a preferred embodiment of the present invention, said source of cellulose has a lignin content of less than 1.5% of the total weight of said cellulose and a hemicellulose content of less than 15% of the total weight of said cellulose. Preferably, said source of cellulose has a lignin content of less than 1.0% of the total weight of said cellulose and a hemicellulose content of less than 10% of the total weight of said cellulose. More preferably, said source of cellulose has a lignin content of less than 0.5% of the total weight of said cellulose and a hemicellulose content of less than 5% of the total weight of said cellulose.

According to a preferred embodiment of the present invention, said solution added to said mixing vessel containing said source of cellulose is a chemical able to degrade cellulose and hemicellulose. Preferably, said solution comprises an acid or base able to degrade cellulose and hemicellulose. More preferably, said solution comprises an acid selected from the group consisting of: sulfuric acid; hydrochloric acid; phosphoric acid; nitric acid; and/or combinations thereof.

According to a preferred embodiment of the present invention, said mixing apparatus contains a horizontal single helicoid auger flight. According to another preferred embodiment of the present invention, said mixing apparatus contains a horizontal double helicoid auger flight.

According to a preferred embodiment of the present invention, said mixing apparatus contains a vertical single helicoid auger flight. According to another preferred embodiment of the present invention, said mixing apparatus contains a vertical double helicoid auger flight.

According to a preferred embodiment of the present invention, said mixing apparatus contains a horizontal single ribbon auger flight. According to another preferred embodiment of the present invention, said mixing apparatus contains a horizontal double ribbon auger flight.

According to a preferred embodiment of the present invention, said mixing apparatus contains a vertical single ribbon auger flight. According to another preferred embodiment of the present invention, said mixing apparatus contains a vertical double ribbon auger flight.

According to a preferred embodiment of the present invention, said mixing apparatus contains a horizontal single sectional auger flight. According to another preferred embodiment of the present invention, said mixing apparatus contains a horizontal double sectional auger flight. According to yet another preferred embodiment of the present invention, said mixing apparatus contains a vertical single sectional auger flight. According to yet another preferred embodiment of the present invention, said mixing apparatus contains a vertical double sectional auger flight.

According to a preferred embodiment of the present invention, said acid is employed at a concentration that is able to break down the glycosidic bonds in cellulose and hemicellulose. Preferably, said acid is employed at a concentration up to 50% wt. of the reaction mixture. More preferably, said acid is employed at a concentration up to 20% wt. of the reaction mixture. Even more preferably, said acid is employed at a concentration up to 10% wt. of the reaction mixture.

It is known to those skilled in the art that the pH of said resulting mixture will be alkaline or acidic depending on the type of solution employed. According to an aspect of the present invention, said resulting mixture may be neutralized for subsequent processing to a pH ranging between 3.0 to 8.0.

According to a preferred embodiment of the present invention, the temperature of the reaction vessel when mixing the reaction mixture may be increased to accelerate the degradation of cellulose and hemicellulose. In some aspects of the present invention, the temperature of that step occurs at a temperature lower than 150° C. In a more preferred aspect of the present invention, the temperature of the mixing step occurs of a temperature lower than 120° C. In a more preferred aspect of the present invention, the temperature of the mixing step occurs of a temperature lower than 100° C. In an even more preferred aspect of the present invention, the temperature of the mixing step occurs of a temperature lower than 80° C.

According to an aspect of the present invention, there is provided a process for the liquefaction of cellulose, said process comprising:
providing a source of cellulose; wherein said source of cellulose comprises cellulose and hemicellulose, wherein said source of cellulose is non-flowable;
adding said source of cellulose to a mixing vessel; wherein said mixing vessel comprises a mixing apparatus selected from the group consisting of: a horizontal single helicoid auger flight; a horizontal double helicoid auger flight; a vertical single helicoid auger flight; a vertical double helicoid auger flight; a horizontal single ribbon auger flight; a horizontal double ribbon auger flight; a vertical single ribbon auger flight; a vertical double ribbon auger flight; a horizontal single sectional auger flight: a horizontal double sectional auger flight; and a vertical single sectional auger flight; a vertical double sectional auger flight;
adding a buffer solution to said mixing vessel containing said source of cellulose to yield a reaction mixture where said source of cellulose is present in a concentration of solids ranging from 5% to 50% of the total mass of said reaction mixture; and wherein said reaction mixture is non-flowable;
adding an enzyme mixture to said reaction mixture;
mixing said reaction mixture in said mixing vessel until said reaction mixture has undergone liquefaction to yield a resulting mixture comprising oligomers and monomers from the degradation of cellulose and hemicellulose and wherein said resulting mixture has a viscosity of no more than 15 Pa's; and
optionally, separating said resulting mixture into a liquid stream used for subsequent processing and a solid stream that is introduced back into the mixing vessel for further processing.

Within the context of this invention, the term “non-flowable” associated with a resulting mixture of a high purity cellulose refers to the characteristic of not being easily moved, manipulated, or transported effectively from one vessel to another without the need of complex machinery, manual intervention and/or convoluted steps which have an impact on the cost effectiveness of a large scale commercial operation. The term “non-flowable” also refers to mixtures that exhibit a solid behaviour. The term “flowable” refers to a substrate or mixture that have fluid-like characteristics and therefore can be transported easily from one vessel to another such as with a pump. In the context of the present invention, flowable mixtures have viscosities of no more than 15 Pa·s. Preferably, flowable mixtures have viscosities of no more than 10 Pa·s. Even more preferably, flowable mixtures have viscosities of no more than 6 Pa·s.

It is known to those skilled in the art that to for solid-liquid mixtures, a certain viscosity threshold cannot be reached; otherwise, pumping and transferring of the material becomes cumbersome and expensive. It is known to those skilled in the art that viscosity, typically measured in pascal-seconds (Pas), quantifies a fluid's resistance to flow. By way of example, water, with a viscosity of approximately 0.001 Pa's, flows freely and easily; however, thick cream or yogurt with viscosities close to 5 Pas, flow with more difficulty and only under force. On the other hand, bitumen has a viscosity around 100,000 Pas at room temperature, giving it a tar-like, almost immovable consistency. This stark contract in viscosities highlights how the flow behaviour of substances can differ vastly. According to a preferred embodiment of the present invention, said resulting mixture has a viscosity of no more than 10 Pa·s. Preferably the resulting mixture has a viscosity of no more than 10 Pa·s. More preferably, said resulting mixture has a viscosity of no more than 6 Pa·s.

According to a preferred embodiment of the present invention, said enzyme mixture in an amount of approximately 0.01 to 1 wt % protein per gram of said source of cellulose. Preferably, said enzyme mixture in an amount of approximately 0.02 to 0.5 wt % protein per gram of said source of cellulose. More preferably, said enzyme mixture in an amount of approximately 0.04 to 0.4 wt % protein per gram of said source of cellulose. Preferably, said enzyme mixture comprises at least one cellulase. More preferably, said enzyme mixture further comprises at least one hemicellulase. Even more preferably, said enzyme mixture comprises at least one exo-glucanase, at least one endo-glucanase and at least one β-glucosidase. Yet even more preferably, the enzyme blend comprises at least one exo-glucanase, at least one endo-glucanase; at least one β-glucosidase, at least one endo-xylanase; and at least one β-xylosidase.

According to a preferred embodiment of the present invention, said reaction mixture is heated to a temperature of up to 70° C. prior to being added to said mixing vessel. According to another preferred embodiment of the present invention, said reaction mixture is heated to a temperature of up to 70° C. during said mixing step. According to more preferred embodiment of the present invention, said reaction mixture is heated to a temperature of up to 55° C. during said mixing step.

Preferably, said buffer solution has a pH ranging from about 3.0 to 8.0, more preferably, said buffer solution has a pH ranging from about 4.0-6.0.

According to a preferred embodiment of the present invention, there is provided a process for the liquefaction of cellulose, said process comprising:
providing a source of cellulose, wherein said source of cellulose has a lignin content of less than 1% of the total weight of said cellulose and a hemicellulose content of less than 15% of the total weight of said cellulose, wherein said source of cellulose is non-flowable;
adding said source of cellulose to a mixing vessel;
adding a solution to said mixing vessel containing said source of cellulose to yield a reaction mixture wherein said source of cellulose is present in a concentration of solids ranging from 5% to 50% of the total mass of said reaction mixture;
mixing said reaction mixture in said mixing vessel until said reaction mixture has undergone liquefaction to yield a resulting mixture comprising oligomers and monomers from the degradation of cellulose and hemicellulose and wherein said resulting mixture has a viscosity of no more than 15 Pa·s;
optionally, separating said resulting mixture into a liquid stream used for subsequent processing and a solid stream that is introduced back into the mixing vessel for further processing.

According to a preferred embodiment of the present invention, there is provided a process for the liquefaction of cellulose, said process consisting of:
providing a source of cellulose, wherein said source of cellulose has a lignin content of less than 1% of the total weight of said cellulose and a hemicellulose content of less than 15% of the total weight of said cellulose; wherein said source of cellulose is non-flowable;
adding said source of cellulose to a mixing vessel;
adding a solution to said mixing vessel containing said source of cellulose to yield a reaction mixture wherein said source of cellulose is present in a concentration of solids ranging from 5% to 50% of the total mass of said reaction mixture;
mixing said reaction mixture in said mixing vessel until said reaction mixture has undergone liquefaction to yield a resulting mixture comprising oligomers and monomers from the degradation of cellulose and hemicellulose and wherein said resulting mixture has a viscosity of no more than 15 Pa·s;
optionally, separating said resulting mixture into a liquid stream used for subsequent processing and a solid stream that is introduced back into the mixing vessel for further processing.

According to a preferred embodiment of the present invention, said reaction mixture is non-flowable at the start of the mixing step.

According to a preferred embodiment of the present invention, there is provided a process for the liquefaction of cellulose, said process comprising:
providing a source of cellulose, wherein said source of cellulose has a lignin content of less than 1% of the total weight of said cellulose and a hemicellulose content of less than 15% of the total weight of said cellulose, wherein said source of cellulose is non-flowable;
adding said source of cellulose to a mixing vessel;
adding a buffer solution to said mixing vessel containing said source of cellulose to yield a reaction mixture where said source of cellulose is present in a concentration of solids ranging from 5% to 50% of the total mass of said reaction mixture; and wherein said reaction mixture is non-flowable;
adding an enzyme mixture to said reaction mixture;
mixing said reaction mixture in said mixing vessel until said reaction mixture has undergone liquefaction to yield a resulting mixture comprising oligomers and monomers from the degradation of cellulose and hemicellulose and wherein said resulting mixture has a viscosity of no more than 15 Pa·s;
optionally, separating said resulting mixture into a liquid stream used for subsequent processing and a solid stream that is introduced back into the mixing vessel for further processing.

According to a preferred embodiment of the present invention, there is provided a process for the liquefaction of cellulose, said process consisting of:
providing a source of cellulose, wherein said source of cellulose has a lignin content of less than 1% of the total weight of said cellulose and a hemicellulose content of less than 15% of the total weight of said cellulose; wherein said source of cellulose is non-flowable;
adding said source of cellulose to a mixing vessel;
adding a buffer solution to said mixing vessel containing said source of cellulose to yield a reaction mixture where said source of cellulose is present in a concentration of solids ranging from 5% to 50% of the total mass of said reaction mixture; and wherein said reaction mixture is non-flowable;
adding an enzyme mixture to said reaction mixture;
mixing said reaction mixture in said mixing vessel until said reaction mixture has undergone liquefaction to yield a resulting mixture comprising oligomers and monomers from the degradation of cellulose and hemicellulose and wherein said resulting mixture has a viscosity of no more than 15 Pa·s.
optionally, separating said resulting mixture into a liquid stream used for subsequent processing and a solid stream that is introduced back into the mixing vessel for further processing.

It is known to the person skilled in the art that various value-added products may be produced and maximized based on the process described herein. Various value-added products may be obtained from the fermentation or conversion of the resulting mixture, which is rich is sugars. The different value-added products are obtained when different fermenting organisms or reaction conditions are employed. Examples of value-added products obtained from the fermentation of the hydrolysate obtained in the present invention include but are not limited to organic acids (i.e., formic acid, acetic acid), alcohols (i.e., ethanol, isopropanol, isobutanol, n-butanol, propanol), ketones (i.e., acetone), and combinations thereof. In a preferred embodiment of the present invention, the value-added product is ethanol. Examples of value-added products obtained from the conversion of the hydrolysate obtained in the present invention include but are not limited to furan-based derivatives (i.e., furfural, 5-hydroxymethyl-furfural, etc.), organic acids such as levulinic acid, sugar alcohols, etc.

### BRIEF DESCRIPTION OF THE FIGURES

The invention may be more completely understood in consideration of the following description of various embodiments of the invention in connection with the accompanying figure, in which:

FIG. 1 is a graph of the percent solids and sugar content over time of a high purity cellulose obtained from the delignification of hardwood biomass undergoing liquefaction in a screw auger according to a preferred embodiment of the process of the present invention;

FIG. 2 is a graph of the percent conversion of cellulose to ethanol over time of SSF reactions performed on high purity cellulose at 5% wt. loading and at 15% wt. loading after liquefaction using a method and reactor described herein;

FIG. 3 is a graph of the percent solids and sugar content over time of canola straw biomass undergoing liquefaction in a screw auger according to a preferred embodiment of the process of the present invention;

FIG. 4 is a graph depicting the mixer content and viscosity over time for a high-purity cellulose slurry undergoing enzymatic liquefaction at 0.19% protein per gram of the weight of cellulosic solids, in a mixing vessel according to a preferred embodiment of the present invention;

FIG. 5 is a graph depicting the mixer content and viscosity over time for a high-purity cellulose slurry undergoing enzymatic liquefaction at 0.09% protein per gram of the weight of cellulosic solids, in a mixing vessel according to a preferred embodiment of the present invention;

FIG. 6 is a graph depicting the mixer content and viscosity over time for a high-purity cellulose slurry undergoing enzymatic liquefaction at 0.05% protein per gram of the weight of cellulosic solids, in a mixing vessel according to a preferred embodiment of the present invention;

FIG. 7 is a graph depicting the mixer content over time for a canola straw slurry undergoing enzymatic liquefaction in a mixing vessel according to a preferred embodiment of the present invention;

FIG. 8 is a graph depicting the mixer content and viscosity over time for a high-purity cellulose slurry undergoing the first stage of enzymatic liquefaction in a mixing vessel according to a preferred embodiment of the present invention;

FIG. 9 is a graph depicting the mixer content and viscosity over time for a high-purity cellulose slurry undergoing the first stage of enzymatic liquefaction and second stage of saccharification in a mixing vessel according to a preferred embodiment of the present invention;

FIG. 10 is a graph depicting the mixer content and viscosity over time for a high-purity cellulose slurry undergoing a chemical liquefaction in a mixing vessel according to a preferred embodiment of the present invention;

FIG. 11 is a graph depicting the mixer content over time for a canola straw slurry undergoing chemical liquefaction in a mixing vessel according to a preferred embodiment of the present invention;

FIG. 12 is a graph depicting the mixer content and viscosity over time for a high-purity cellulose slurry after a chemical liquefaction in a mixing vessel according to a preferred embodiment of the present invention, neutralized and then transferred to a CSTR to undergo enzymatic saccharification according to a preferred embodiment of the present invention; and

FIG. 13 displays the process described herein in the context of the production of ethanol.

### DETAILED DESCRIPTION OF EMBODIMENTS OF THE PRESENT INVENTION

The description that follows, and the embodiments described therein, is provided by way of illustration of an example, or examples, of particular embodiments of the principles of the present invention. These examples are provided for the purposes of explanation, and not limitation, of those principles and of the invention.

According to a preferred embodiment of the present invention, the process to liquefy a high solids content reaction mixture comprises two main steps: the delignification of a biomass comprising cellulose and liquefaction of a resulting high purity cellulose into a mixture of depolymerized cellulose. Said mixture of depolymerized cellulose can then subsequently be converted to other chemicals, such as, but not limited to, ethanol.

### Process to Obtain a High Purity Cellulose

According to a preferred embodiment of the present invention, the method of delignification of a lignocellulosic biomass material which yields a high purity cellulose (may also be referred to as modified Caro's acid delignified cellulose) comprise:
providing a biomass material comprising cellulose fibers and lignin;
exposing said biomass material requiring delignification to a modified Caro's acid composition having a pH of less than 1, said modified Caro's acid composition selected from the group consisting of: composition A; composition B; composition C; composition D; composition E; composition F; composition G; composition H; composition I; and composition J;
wherein said composition A comprises:
   sulfuric acid;
   a compound comprising an amine moiety and a sulfonic acid moiety; and
   a peroxide; and wherein sulfuric acid, said compound comprising an amine moiety and a sulfonic acid moiety and said peroxide are present in a molar ratio of no less than 1:1:1;
wherein said composition B comprises:
   sulfuric acid;
   a compound comprising an amine moiety;
   a compound comprising a sulfonic acid moiety; and
   a peroxide; wherein sulfuric acid and said a compound comprising an amine moiety and said compound comprising a sulfonic acid moiety are present in a molar ratio of no less than 1:1:1;
wherein said composition C comprises:
   an alkylsulfonic acid; and
   a peroxide; wherein said alkylsulfonic acid and said peroxide are present in a molar ratio of no less than 1:1;
wherein said composition D comprises:
   O sulfuric acid;
   a heterocyclic compound; and
   a peroxide; and wherein sulfuric acid and said a heterocyclic compound; are present in a molar ratio of no less than 1:1;
wherein said composition E comprises:
   sulfuric acid;
   a modifying agent comprising a compound containing an amine group; and
   a peroxide; and wherein sulfuric acid and said compound containing an amine group; are present in a molar ratio of no less than 1:1;
wherein said composition F comprises:
   sulfuric acid;
   a modifying agent comprising an alkanesulfonic acid and
   a peroxide; and wherein sulfuric acid and said alkanesulfonic acid are present in a molar ratio of no less than 1:1;
wherein said composition G comprises:
   sulfuric acid;
   a substituted aromatic compound; and
   a peroxide; and wherein sulfuric acid and said substituted aromatic compound; are present in a molar ratio of no less than 1:1;
wherein said composition H comprises:
   sulfuric acid;
   a modifying agent comprising an arylsulfonic acid;
   O a peroxide; and
   optionally, a compound containing an amine group; wherein sulfuric acid and said a arylsulfonic acid; are present in a molar ratio of no less than 1:1;
wherein said composition I comprises:
   sulfuric acid;
   a heterocyclic compound;
   an alkanesulfonic acid and
   a peroxide; and wherein sulfuric acid and said a heterocyclic compound; are present in a molar ratio of no less than 1:1;
wherein said composition J comprises:
   sulfuric acid;
   a carbonyl-containing nitrogenous base compound; and
   a peroxide; and wherein sulfuric acid and said a carbonyl-containing nitrogenous base compound; are present in a molar ratio of no less than 1:1;
for a period of time sufficient to remove substantially all of the lignin present on said lignocellulosic biomass material. The process can be carried out for a varying duration of time depending on the particle size of the biomass being fed into the process. The process can last from 2 to 20 hours depending on that characteristic. Moreover, the temperature of the resulting mixture also has an impact on the duration of the process as the reaction is highly exothermic, precautions are taken to prevent a runaway degradation of the cellulose. This would result in a carbon black resulting product with no value. The process is preferably run at temperatures below 50° C., more preferably at temperatures below 40° C. The process of delignification is preferably performed with a cooling means adapted to control the heat generated by the chemical reaction of delignification and maintain the temperature to avoid an undesirable ‘runaway’ reaction.

Preferably, said sulfuric acid, said compound comprising an amine moiety and a sulfonic acid moiety and said peroxide are present in a molar ratio of no more than 15:1:1. Preferably, for a modified Caro's acid comprising sulfuric acid, peroxide and taurine (as the modifier component), the molar composition is as follows: H₂O:H₂O₂:H₂SO₄:Taurine in a molar ratio of 56:10:10:1. Preferably, for a modified Caro's acid comprising TEOA/MSA, the molar composition is as follows: H₂O:H₂O₂:H₂SO₄:TEOA:MSA in a molar ratio of 56:10:10:1:1.

According to a preferred embodiment of the approach to obtain low lignin cellulose, said sulfuric acid and said compound comprising an amine moiety and a sulfonic acid moiety are present in a molar ratio of no less than 3:1.

Preferably, said compound comprising an amine moiety and a sulfonic acid moiety is selected from the group consisting of taurine; taurine derivatives; and taurine-related compounds. According to a preferred embodiment of the approach to obtain low lignin cellulose, said taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates; as well as aminoalkylsulfonic acids where the alkyl is selected from the group consisting of C₁-C₅ linear alkyl and C₁-C₅ branched alkyl.

Preferably, said linear alkylaminosulfonic acid is selected form the group consisting of: methyl; ethyl (taurine); propyl; and butyl.

Preferably, branched aminoalkylsulfonic acid is selected from the group consisting of: isopropyl; isobutyl; and isopentyl.

According to a preferred embodiment of the approach to obtain low lignin cellulose, said compound comprising an amine moiety and a sulfonic acid moiety is taurine.

According to a preferred embodiment of the approach to obtain low lignin cellulose, said sulfuric acid and compound comprising an amine moiety and a sulfonic acid moiety are present in a molar ratio of no less than 3:1.

According to a preferred embodiment of the approach to obtain low lignin cellulose, said compound comprising an amine moiety is an alkanolamine is selected from the group consisting of: monoethanolamine; diethanolamine; triethanolamine; and combinations thereof.

According to a preferred embodiment of the approach to obtain low lignin cellulose, said compound comprising a sulfonic acid moiety is selected from the group consisting of: alkylsulfonic acids; arylsulfonic acids; and combinations thereof.

Preferably, said alkylsulfonic acid is selected from the group consisting of: alkylsulfonic acids where the alkyl groups range from C₁-C₆ and are linear or branched; and combinations thereof. More preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof.

Preferably, said arylsulfonic acid is selected from the group consisting of: toluenesulfonic acid; benzesulfonic acid; and combinations thereof.

According to a preferred embodiment of the approach to obtain low lignin cellulose, said alkylsulfonic acid; and said peroxide are present in a molar ratio of no less than 1:1.

Preferably, said compound comprising a sulfonic acid moiety is methanesulfonic acid.

According to a preferred embodiment of the approach to obtain source of high purity cellulose (a cellulose having a lignin content of less than 2% of the total weight of said cellulose and having a hemicellulose content of less than 15% of the total weight of said cellulose), said Composition C may further comprise a compound comprising an amine moiety. Preferably, the compound comprising an amine moiety has a molecular weight below 300 g/mol. Preferably also, the compound comprising an amine moiety is a primary amine. More preferably, the compound comprising an amine moiety is an alkanolamine. Preferably, the compound comprising an amine moiety is a tertiary amine. According to a preferred embodiment of the approach to obtain low lignin cellulose, the alkanolamine is selected from the group consisting of: monoethanolamine; diethanolamine; triethanolamine; and combinations thereof. Preferably, the alkanolamine is triethanolamine.

According to a preferred embodiment of the approach to obtain low lignin cellulose, said in Composition C, said sulfuric acid and said a compound comprising an amine moiety and said compound comprising a sulfonic acid moiety are present in a molar ratio of no less than 1:1:1.

Preferably, in Composition C, said sulfuric acid, said compound comprising an amine moiety and said compound comprising a sulfonic acid moiety are present in a molar ratio ranging from 28:1:1 to 2:1:1.

Preferably, in Composition C, said compound comprising an amine moiety is triethanolamine and said compound comprising a sulfonic acid moiety is methanesulfonic acid.

According to preferred embodiment of the present invention, the modified Caro's acid (as disclosed in Canadian patent application 3,128,678) comprises: sulfuric acid; a heterocyclic compound and a peroxide; and wherein sulfuric acid and said a heterocyclic compound; are present in a molar ratio of no less than 1:1. Preferably, the sulfuric acid and said heterocyclic compound are present in a molar ratio ranging from 28:1 to 2:1 More preferably, the sulfuric acid and heterocyclic compound are present in a molar ratio ranging from 24:1 to 3:1. Preferably, the sulfuric acid and heterocyclic compound are present in a molar ratio ranging from 20:1 to 4:1. More preferably, the sulfuric acid and heterocyclic compound are present in a molar ratio ranging from 16:1 to 5:1. Preferably, the sulfuric acid and heterocyclic compound are present in a molar ratio ranging from 12:1 to 6:1. Also preferably, said heterocyclic compound has a molecular weight below 300 g/mol. Also preferably, said heterocyclic compound has a molecular weight below 150 g/mol. More preferably, said heterocyclic compound is a secondary amine. According to a preferred embodiment of the present invention, said heterocyclic compound is selected from the group consisting of: imidazole; triazole; and N-methylimidazole.

According to preferred embodiment of the present invention, the modified Caro's acid (as disclosed in Canadian patent application 3,128,677) comprises: sulfuric acid; a modifying agent comprising a compound containing an amine group and a peroxide; and wherein sulfuric acid and said compound containing an amine group; are present in a molar ratio of no less than 1:1. Preferably, the sulfuric acid and said compound containing an amine group are present in a molar ratio ranging from 28:1 to 2:1. More preferably, the sulfuric acid and compound containing an amine group are present in a molar ratio ranging from 24:1 to 3:1. Preferably, the sulfuric acid and compound containing an amine group are present in a molar ratio ranging from 20:1 to 4:1. More preferably, the sulfuric acid and compound containing an amine group are present in a molar ratio ranging from 16:1 to 5:1. Preferably, the sulfuric acid and compound containing an amine group are present in a molar ratio ranging from 12:1 to 6:1. According to a preferred embodiment of the present invention, the modifying agent is selected in the group consisting of: TEOA; MEOA; pyrrolidine; DEOA; ethylenediamine; diethylamine; triethylamine; morpholine; MEA-triazine; and combinations thereof. According to a more preferred embodiment of the present invention, the modifying agent is TEOA; MEOA; pyrrolidine; DEOA; ethylenediamine; triethylamine.

According to preferred embodiment of the present invention, the modified Caro's acid (as disclosed in Canadian patent application 3,128,676) comprises: sulfuric acid; a modifying agent comprising an alkanesulfonic acid and a peroxide; and wherein sulfuric acid and said alkanesulfonic acid are present in a molar ratio of no less than 1:1. Preferably, said alkanesulfonic acid is selected from the group consisting of: alkanesulfonic acids where the alkyl groups range from C₁-C₆ and are linear or branched; and combinations thereof. Preferably, said alkanesulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. More preferably, said alkanesulfonic acid is methanesulfonic acid. Also preferably, said alkanesulfonic acid has a molecular weight below 300 g/mol. Also preferably, said alkanesulfonic acid has a molecular weight below 150 g/mol. Preferably, the sulfuric acid and said alkanesulfonic acid and are present in a molar ratio ranging from 28:1 to 2:1. More preferably, the sulfuric acid and alkanesulfonic acid are present in a molar ratio ranging from 24:1 to 3:1. Preferably, the sulfuric acid and alkanesulfonic acid are present in a molar ratio ranging from 20:1 to 4:1. More preferably, the sulfuric acid and alkanesulfonic acid are present in a molar ratio ranging from 16:1 to 5:1. According to a preferred embodiment of the present invention, the sulfuric acid and alkanesulfonic acid are present in a molar ratio ranging from 12:1 to 6:1.

According to preferred embodiment of the present invention, the modified Caro's acid (as disclosed in Canadian patent application 3,128,675) comprises: sulfuric acid; a substituted aromatic compound and a peroxide; and wherein sulfuric acid and said substituted aromatic compound; are present in a molar ratio of no less than 1:1. Preferably, the substituted aromatic compound comprises at least two substituents. More preferably, at least one substituent is an amine group and at least one of the other substituent is a sulfonic acid moiety. According to a preferred embodiment, the substituted aromatic compound comprises three or more substituent. According to a preferred embodiment of the present invention, the substituted aromatic compound comprises at least a sulfonic acid moiety. According to another preferred embodiment of the present invention, the substituted aromatic compound comprises an aromatic compound having a sulfonamide substituent, where the compound can be selected from the group consisting of: benzenesulfonamides; toluenesulfonamides; substituted benzenesulfonamides; and substituted toluenesulfonamides. Preferably, the sulfuric acid and said substituted aromatic compound and are present in a molar ratio ranging from 28:1 to 2:1. More preferably, the sulfuric acid and substituted aromatic compound are present in a molar ratio ranging from 24:1 to 3:1. Preferably, the sulfuric acid and substituted aromatic compound are present in a molar ratio ranging from 20:1 to 4:1. More preferably, the sulfuric acid and substituted aromatic compound are present in a molar ratio ranging from 16:1 to 5:1. Preferably, the sulfuric acid and substituted aromatic compound are present in a molar ratio ranging from 12:1 to 6:1.

According to preferred embodiment of the present invention, the modified Caro's acid (as disclosed in Canadian patent application 3,128,674) comprises: sulfuric acid; a modifying agent comprising an arylsulfonic acid; a peroxide; and optionally, a compound containing an amine group; wherein sulfuric acid and said a arylsulfonic acid; are present in a molar ratio of no less than 1:1. Preferably, the compound containing an amine group is selected from the group consisting of: imidazole; N-methylimidazole; triazole; monoethanolamine (MEOA); diethanolamine (DEOA); triethanolamine (TEOA); pyrrolidine and combinations thereof. According to a preferred embodiment of the present invention, sulfuric acid and the peroxide are present in a molar ratio of approximately 1:1. Preferably, the sulfuric acid and said arylsulfonic acid and are present in a molar ratio ranging from 28:1 to 2:1. More preferably, the sulfuric acid and arylsulfonic acid are present in a molar ratio ranging from 24:1 to 3:1. Preferably, the sulfuric acid and arylsulfonic acid are present in a molar ratio ranging from 20:1 to 4:1. More preferably, the sulfuric acid and arylsulfonic acid are present in a molar ratio ranging from 16:1 to 5:1. According to a preferred embodiment of the present invention, the sulfuric acid and arylsulfonic acid are present in a molar ratio ranging from 12:1 to 6:1. Also preferably, said arylsulfonic acid has a molecular weight below 300 g/mol. Also preferably, said arylsulfonic acid has a molecular weight below 150 g/mol. Even more preferably, said arylsulfonic acid is selected from the group consisting of: orthanilic acid; metanilic acid; sulfanilic acid; toluenesulfonic acid; benzenesulfonic acid; and combinations thereof.

According to preferred embodiment of the present invention, the modified Caro's acid (as disclosed in Canadian patent application 3,128,673) comprises: sulfuric acid; a heterocyclic compound; an alkanesulfonic acid and a peroxide; and wherein sulfuric acid and said a heterocyclic compound; are present in a molar ratio of no less than 1:1. Preferably, said aqueous acidic composition comprising: sulfuric acid; a heterocyclic compound; an arylsulfonic acid; and wherein sulfuric acid and said a heterocyclic compound; are present in a molar ratio of no less than 1:1. Preferably, the arylsulfonic acid is toluenesulfonic acid.

Preferably, the sulfuric acid, the heterocyclic compound and the alkanesulfonic acid are present in a molar ratio ranging from 28:1:1 to 2:1:1. More preferably, the sulfuric acid the heterocyclic compound and the alkanesulfonic acid are present in a molar ratio ranging from 24:1:1 to 3:1:1. Preferably, the sulfuric acid, the heterocyclic compound and the alkanesulfonic acid are present in a molar ratio ranging from 20:1:1 to 4:1:1. More preferably, the sulfuric acid, the heterocyclic compound and the alkanesulfonic acid are present in a molar ratio ranging from 16:1:1 to 5:1:1. According to a preferred embodiment of the present invention, the sulfuric acid and heterocyclic compound are present in a molar ratio ranging from 12:1:1 to 6:1:1. Also preferably, said heterocyclic compound has a molecular weight below 300 g/mol. Also preferably, said heterocyclic compound has a molecular weight below 150 g/mol. Even more preferably, said heterocyclic compound is selected from the group consisting of: imidazole; triazole; n-methylimidazole; and combinations thereof. Preferably, the alkanesulfonic acid is selected from the group consisting of: alkylsulfonic acids where the alkyl groups range from C₁-C₆ and are linear or branched; and combinations thereof. Preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. More preferably, said alkylsulfonic acid is methanesulfonic acid.

According to preferred embodiment of the present invention, the modified Caro's acid (as disclosed in Canadian patent application 3,128,672) comprises: sulfuric acid; a carbonyl-containing nitrogenous base compound and a peroxide; and wherein sulfuric acid and said a carbonyl-containing nitrogenous base compound; are present in a molar ratio of no less than 1:1. According to a preferred embodiment of the present invention, the carbonyl-containing nitrogenous base compound is selected from the group consisting of: caffeine; lysine; creatine; glutamine; creatinine; 4-aminobenzoic acid; glycine; NMP (N-methyl-2-pyrrolidinone); histidine; DMA (N,N-dimethylacetamide); arginine; 2,3-pyridinedicarboxylic acid; hydantoin; and combinations thereof. Preferably, the sulfuric acid and said carbonyl-containing nitrogenous base compound and are present in a molar ratio ranging from 28:1 to 2:1. More preferably, the sulfuric acid and carbonyl-containing nitrogenous base compound are present in a molar ratio ranging from 24:1 to 3:1. Preferably, the sulfuric acid and carbonyl-containing nitrogenous base compound are present in a molar ratio ranging from 20:1 to 4:1. More preferably, the sulfuric acid and carbonyl-containing nitrogenous base compound are present in a molar ratio ranging from 16:1 to 5:1. According to a preferred embodiment of the present invention, the sulfuric acid and carbonyl-containing nitrogenous base compound are present in a molar ratio ranging from 12:1 to 6:1.

According to another aspect of the present invention, by combining sugar and starch materials with a high purity cellulose, as defined above, and fermenting this combined blend, one can generates a value added product such as ethanol. Said high purity cellulose being defined as having a low kappa number and low hemicellulose content.

Preferably, the combination of a sugar stream from each process significantly decreases the carbon intensity score of the value-added product generated when compared to the process where only the sugar or starch material is employed.

### Experiment #1

A high purity cellulose obtained from the delignification of hardwood biomass using a modified Caro's acid was used for this experiment. The delignification was run for 6 hours at temperatures between 35 and 42° C., after which the cellulosic solids were separated from the liquid stream using a filter press and neutralized to a pH 6-7 using NaOH. The cellulose was characterized prior to exposing it to the liquefaction step. The characterization is found in Table 1. The Kappa number was measured as TAPPI method T236. Ash content was measured as per TAPPI method T211, and the distribution of cellulose and hemicellulose content was determined as per TAPPI method T203.

TABLE 1
Characterization of the cellulosic solids obtained from the delignification
of hardwood biomass used in the liquefaction experiment.

| Parameter | Result |
|---|---|
| Kappa number | 1.3 |
| Lignin content (% wt.) | 0.17% |
| Ash content (% wt.) | 4.17% |
| Total C (Cellulose and hemicellulose content (% wt.)) | 95.66% |
| Cellulose content (% wt.) in Total C | 92.9% |
| Hemicellulose content (% wt.) in Total C | 7.1% |

A helicoidal mixing reactor utilized was constructed with stainless steel SS304. The length of the reactor was 167″, the total height was 12″, with a U shape trough. This unit was fully sealed with a series of 4 removable lids. The volume of the reactor was 120 L. The auger pitch was 5.5″ and the diameter of the shaft was 3″, the overall screw diameter is 9″ and used a helix flight. The auger was mechanically rotated by a 2 hp motor at 3600 rpm with a 40:1 gear ratio. The unit was equipped with a variable frequency drive. This allows the rotation to be slowed down to 1-2 rpm, which equates to a residence time of 10 minutes. To ensure product in the reactor had sufficient residence time, every 10 mins there was a rotation change done via the variable frequency drive (VFD). The heating in the screw reactor was done using a ¾″ to 1″ pipe jacket which transferred heat via convection and conduction. To reduce heat loss, the screw reactor was then insulated using R22 wool insulation and foil insulation.

A 100 mM citrate buffer (pH 5.3) was prepared and pumped into the mixing reactor equipped with a screw auger. A cellulase enzyme cocktail was added to the reactor in an amount equivalent to 3-4 mg protein per gram of the weight of dry cellulose. The cellulosic material was added as a never dried product (which comes directly from the delignification after washing but no drying step), containing approximately 50% cellulose w/w into the reactor, to obtain a dry cellulose loading of 15% w/w of the entire reaction mixture (buffer, enzyme and cellulose).

The slurry was heated up to a temperature of 49° C. and the auger in the screw reactor was rotated at 2 revolutions per minute, changing direction every 10 minutes. Samples were collected every 4 hours for a period of 24 hours, beginning when the slurry was homogeneous (approximately 30 minutes of mixing after materials addition). Two samples were collected at each time point from access hatches on opposite ends of the reactor. Each sample was then rinsed twice at a 2:1 ratio of distilled water to sample. The filtrate was collected for sugar analysis via HPLC, and the cellulose cake (solids) was dried for percent solids determination.

### Results

Percent solids was measured on the rinsed slurry samples obtained over a 24-hour time period. These measurements help quantify the amount of cellulose remaining in the reactor that has not been degraded to small molecular weight oligosaccharides that are soluble in water and thus, rinsed through the rinsing step.

Samples taken on opposite ends of the screw reactor from different access hatches were averaged to obtain a mean percent solid of the reaction mixture for that time point. Initial percent solids content after 30 minutes was determined to be 17.4% wt. while after 24 hours of hydrolysis, the percent solids decreased to 10.0% wt., an amount that makes the slurry pumpable into subsequent steps of the conversion to ethanol and other high value-added products (Table 2 and FIG. 1). Some variation was observed between samples of the same time point, which is likely due to reaction slurry heterogeneity.

TABLE 2
Results from the liquefaction step performed in Experiment 1

| | | % wt. | Glucose | Total sugar |
|---|---|---|---|---|
| Time point | | solids | content (kg) | content (kg) |
| Initial timepoint | | 17.4 ± 0.2 | 1.3 ± 0.2 | 1.7 ± 0.2 |
| (30 minutes after initiating | | | | |
| mixing of the reaction mixture) | | | | |
| 4 | hours | 13.3 ± 0.7 | 2.2 ± 0.1 | 2.6 ± 0.1 |
| 8 | hours | 14.4 ± 2.4 | 3.2 ± 0.2 | 3.9 ± 0.2 |
| 12 | hours | 11.8 ± 0.8 | 3.6 ± 0.2 | 4.3 ± 0.2 |
| 16 | hours | 10.9 ± 0.3 | 3.5 ± 0.4 | 4.1 ± 0.5 |
| 20 | hours | 10.5 ± 0.2 | 4.4 ± 0.1 | 5.2 ± 0.2 |
| 24 | hours | 10.0 ± 0.2 | 4.8 ± 0.3 | 5.7 ± 0.3 |

Using HPLC, the content of glucose and total sugar content (including all C6 and C5 sugars) was measured. Dissolved sugar content rose to 5.68 kg in the screw reactor, with the majority of the sugars being glucose at 4.80 kg (FIG. 1). It is speculated that the remaining difference between the cellulose and sugar content after 24 hours of hydrolysis are larger saccharides composed of three or more monomers that are not quantifiable with the HPLC column in use.

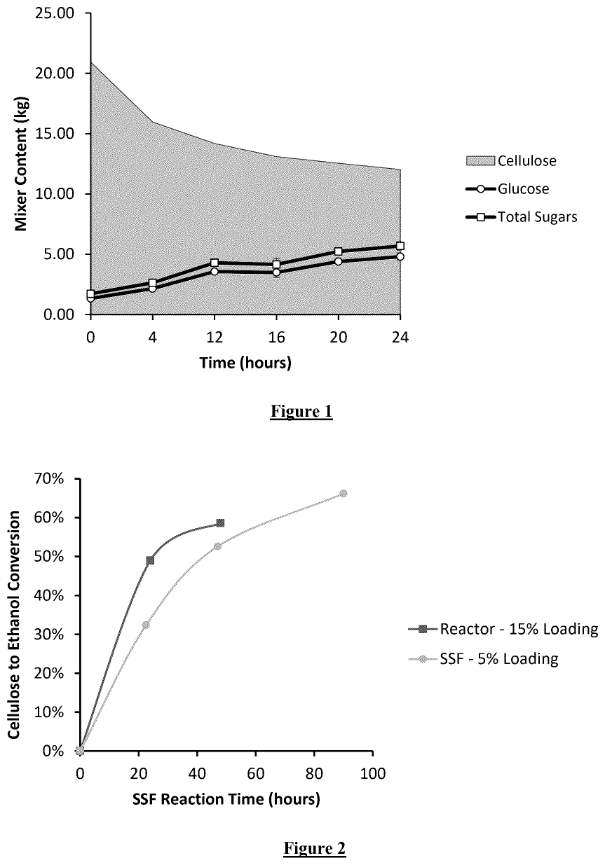

After 24 hours of residence time in the reactor, a fraction of the liquified slurry was transferred to a 10 L reactor, where it underwent a simultaneous saccharification and fermentation (SSF) reaction. The slurry was spiked with loading of dry Saccharomyces cerevisiae equivalent to 2 g/L and left at 37° C. and stirring at 100 rpm for 113 hours. Maximum ethanol production was observed after 48 hours, with a yield of 6.8 L of ethanol, which represents a conversion of 60% of the total cellulosic solids (added in the mixing reactor) to ethanol. This is comparable to an SSF reaction with the same high purity cellulose, but at 5% wt. cellulose loading and a conversion of 66% of the total cellulosic solids to ethanol after 48 hours (FIG. 2).

It is believed that, by employing a preferred embodiment of the process according to the present invention, the conversion of cellulose to depolymerized (hence liquefied cellulose) can reach up to 80% conversion rate. Advantageously, the proposed process according to a preferred embodiment employs a cellulose which is very low in lignin content as well as hemicellulose. It is well known that the absence hemicellulose in the feedstock to be liquified is preferred

### Experiment #2

The following experimentation was conducted using untreated canola straw as the biomass feedstock. The canola straw was characterized to determine the content of acid-insoluble lignin (also known as Klason lignin) as well as carbohydrate composition. The carbohydrate composition gives an indication of cellulose and hemicellulose distribution within the biomass. Results of the characterization of the canola straw are shown in Table 3.

TABLE 3
Results of the characterization of canola
straw used in the experiments

| Parameter | Units | Result |
|---|---|---|
| Klason lignin | %, OD basis | 16.1% |
| Arabinan | %, extracted OD basis | 1.2% |
| Xylan | %, extracted OD basis | 11.4% |
| Mannan | %, extracted OD basis | 1.0% |
| Galactan | %, extracted OD basis | 1.1% |
| Glucan | %, extracted OD basis | 24.9% |

A 100 mM citrate buffer (pH 5.3) was prepared and pumped into the screw reactor. Canola was added into the reactor at 15% wt. solids loading, to mimic the solids loading of Experiment #1. A cellulase enzyme cocktail was added to the screw reactor in an amount equivalent to 3-4 mg protein per gram of the weight of dry carbohydrate content.

The canola straw biomass was added into the screw reactor, to obtain a canola loading of about 15% w/w of the entire reaction mixture. The slurry was brought up to a temperature of 49° C. and the auger moved throughout the screw reactor at 2 revolutions per minute, switching direction every 10 minutes. Samples were collected every 4 hours for 24 hours, beginning at approximately 30 minutes after materials addition. Two samples were collected at each time point from access hatches on opposite ends of the screw reactor. Samples were then rinsed twice at a 2:1 ratio of distilled water to sample. The filtrate was collected for sugar analysis via HPLC, and the cellulose cake was dried for percent solids determination.

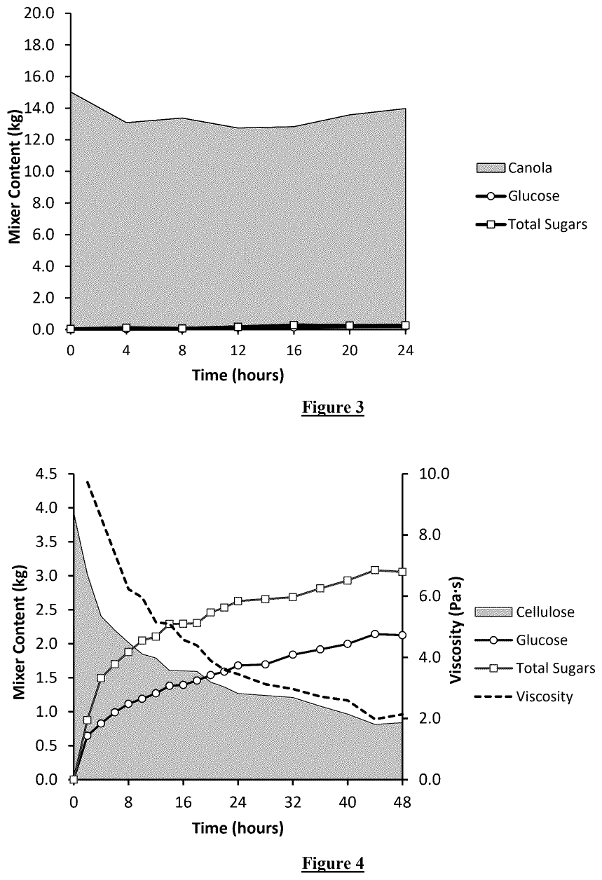

Percent solids was measured on the rinsed slurry samples obtained over a 24-hour time period. These measurements allow to quantify the amount of cellulose remaining in the reactor that has not been degraded to small molecular weight oligosaccharides that are soluble in water and thus, rinsed through the rinsing step. Samples taken on opposite ends of the screw reactor from different access hatches were averaged to obtain a mean percent solid of the reaction mixture for that time point. Initial percent solids content after 30 minutes was determined to be 25.0% wt. There was no obvious change in the percent solids content over the 24 hours of hydrolysis (see FIG. 3 and Table 4). Compared to the cellulose trial, there was more variation between samples of the same time point. This can be explained by the reaction slurry heterogeneity, as the canola straw biomass did not mix well into the buffer solution.

TABLE 4
Results from the liquefaction step performed in Experiment 2

| | | % wt. | Glucose | Total sugar |
|---|---|---|---|---|
| Time point | | solids | content (kg) | content (kg) |
| Initial timepoint | | 25.0 ± 1.1 | 0.02 ± 0.00 | 0.03 ± 0.02 |
| (30 minutes after initiating | | | | |
| mixing of the reaction mixture) | | | | |
| 4 | hours | 21.8 ± 2.9 | 0.03 ± 0.01 | 0.11 ± 0.07 |
| 8 | hours | 22.3 ± 2.0 | 0.04 ± 0.02 | 0.06 ± 0.04 |
| 12 | hours | 21.2 ± 0.1 | 0.12 ± 0.09 | 0.16 ± 0.11 |
| 16 | hours | 21.4 ± 0.1 | 0.14 ± 0.06 | 0.28 ± 0.02 |
| 20 | hours | 22.6 ± 3.2 | 0.19 ± 0.10 | 0.25 ± 0.12 |
| 24 | hours | 23.3 ± 1.5 | 0.20 ± 0.02 | 0.26 ± 0.03 |

Glucose and total sugar content were measured via HPLC, both of which were near negligible amounts. There was a slight increase in the total sugar content over time, reaching up to 0.12 kg (FIG. 3). This is just 2.1% of what was achieved when a cellulose obtained from the delignification of canola straw was used as the feedstock into this liquefaction process. Considering the small amounts of glucose obtained and the high viscosity of the sample, no follow up reactions were conducted after the screw reactor hydrolysis.

For all the following experiments, the mixing vessel used was a horizontal mixer with an exterior jacket capable of cooling or heating the contents inside the vessel. The unit was constructed with stainless steel SS304. The length of the vessel was 20″, the height was 14″, the total width was 12″, with a U shape trough. This unit had a hinged lid with a safety contact switch. The volume of the reactor is 1.2 ft³. The ribbon flight mixer was used with a diameter of the rotating blade was 6″ with a 1″ shaft. The auger was mechanically rotated by a ¾ hp motor at 1800 rpm with an 18:1 gear ratio. The unit was equipped with a variable frequency drive. This allowed the rotation to be slowed down to 1-2 rpm or as much as 99 rpm. The heating in the screw reactor was done using the exterior jacket which used ethylene glycol as a heat transfer fluid. The heat transfer was done via conduction.

### Experiment #3

A high purity cellulose obtained from the delignification of canola biomass using a modified Caro's acid was used for this experiment. After the delignification, cellulosic solids were separated from the liquid stream using a filter press and neutralized to a pH 6-7 using NaOH. The cellulose was characterized prior to exposing it to the liquefaction step. The characterization is found in Table 5. The Kappa number was measured as TAPPI method T236. Ash content was measured as per TAPPI method T211, and the distribution of cellulose and hemicellulose content was determined as per TAPPI method T203.

TABLE 5
Characterization of the cellulosic solids obtained from the delignification
of canola biomass used in the liquefaction experiment.

| Parameter | Result |
|---|---|
| Kappa number | 3.9 |
| Lignin content (% wt.) | 0.58% |
| Cellulose content (% wt.) in Pulp | 89.47% |
| Hemicellulose content (% wt.) in Pulp | 10.53% |

The cellulosic material was added as a “never dried” product to the mixing vessel equipped with interwoven ribbon blades. A citrate buffer concentrate was prepared and pumped into the mixing vessel. The slurry was heated up to a temperature of 50° C. and the auger in the screw reactor was rotated at 22.5 revolutions per minute, changing direction every 30 minutes. The cellulosic material with the buffer was left to distribute homogeneously before taking a t=0 sample. A cellulase enzyme cocktail was then added to the reactor in differing amounts of protein per gram of the weight of cellulosic solids (see Table 6). The amounts of cellulosic material, buffer and enzyme were selected to obtain a dry cellulose loading of 25% wt. of the entire reaction mixture.

TABLE 6
Parameters of each of the reactions carried out in Experiment #3.

| | Experiment | Time of | Enzyme loading |
|---|---|---|---|
| Feedstock | ID | mixing (h) | (% wt. protein) |
| Cellulose | Experiment 3-a | 48 hours | 0.19 |
| Cellulose | Experiment 3-b | 48 hours | 0.09 |
| Cellulose | Experiment 3-c | 16 hours | 0.05 |

Samples were collected every 2 hours for a period of 24 hours, and every 4 hours subsequently until the end of the test. Each sample was then extracted with distilled water. The filtrate was collected for sugar analysis via HPLC, and the cellulosic solids were dried for percent solids determination. This approach helps quantify the amount of cellulose remaining in the reactor that has not been degraded to small molecular weight oligosaccharides that are soluble in water and thus, extracted into the distilled water phase. Viscosity of the samples was measured after a predetermined threshold, when samples were homogeneous. Viscosity was measured using a rotational viscometer.

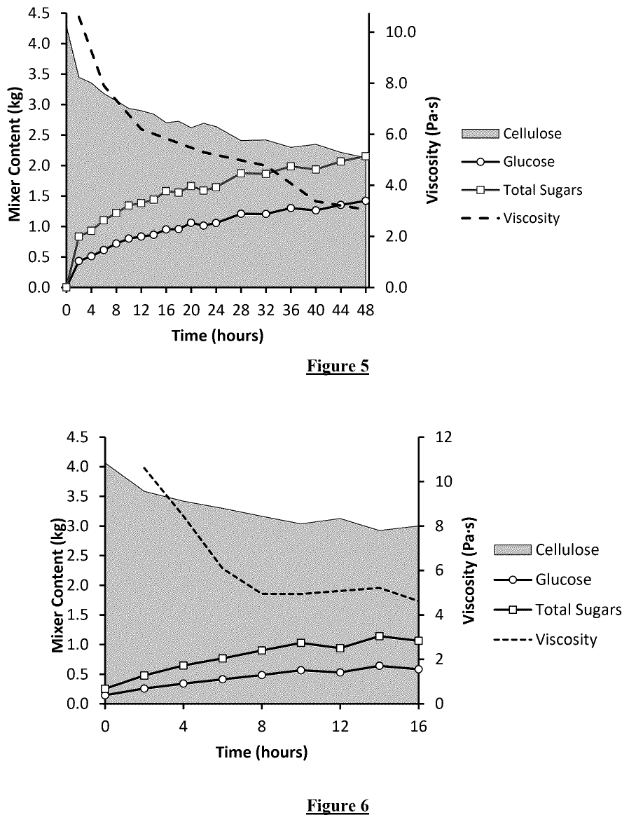

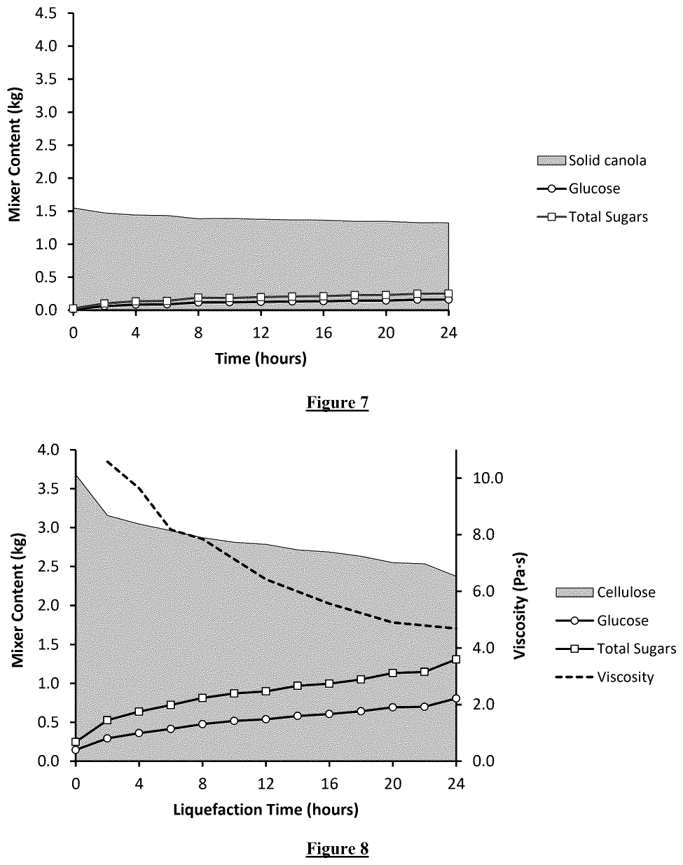

The results of Experiment #1 are shown in FIGS. 4, 5, and 6. As expected, as the liquefaction progressed, the glucose as well as total sugar content (comprising of oligosaccharides, cellobiose, xylose, and arabinose) increased. The viscosity data displayed a sharp decrease in all samples occurring during the first 4-8 hours followed by a period of a less sharp decrease. In all cases, the material post-liquefaction was able to be pumped. As expected, sugar content increased proportionally with the enzyme loading, with the sugar content being 45 and 116% higher in Experiment 3-a with a 0.30% wt. enzymatic protein loading than the other two loadings in Experiment 3-b and Experiment 3-c at the 16 hour timepoint.

The results of this experimentation confirm that the use of a mixing vessel comprising a horizontal ribbon flight allows for the rapid degradation of a high solids content of high purity cellulose, this leads to a sharp decrease in the viscosity of the mixture. Such an experiment confirms the ability to process a larger amount of cellulose solids per unit of time than other known methods.

### Experiment #4

The following experimentation was conducted using untreated canola straw as the biomass feedstock. The canola straw was characterized to determine the content of acid-insoluble lignin (also known as Klason lignin) as well as carbohydrate composition. The carbohydrate composition gives an indication of cellulose and hemicellulose distribution within the biomass. Results of the characterization of the canola straw are shown in Table 7.

TABLE 7
Results of the characterization of
canola straw used in Experiment #4

| Parameter | Units | Result |
|---|---|---|
| Klason lignin | %, OD basis | 16.1% |
| Arabinan | %, extracted OD basis | 1.2% |
| Xylan | %, extracted OD basis | 11.4% |
| Mannan | %, extracted OD basis | 1.0% |
| Galactan | %, extracted OD basis | 1.1% |
| Glucan | %, extracted OD basis | 24.9% |

The canola straw was added to the mixing vessel equipped with interwoven ribbon blades. A citrate buffer concentrate was prepared and pumped into the mixing vessel. The slurry was heated up to a temperature of 50° C. and the auger in the screw reactor was rotated at 22.5 revolutions per minute, changing direction every 30 minutes. The canola straw with the buffer was left to distribute homogeneously before taking a t=0 sample. A cellulase enzyme cocktail was then added to the reactor in a concentration of 0.13% wt. enzymatic protein of dry solids. The amounts of canola straw, buffer and enzyme were selected to obtain a dry canola loading of 25% wt. of the entire reaction mixture. Samples were collected every 2 hours for a period of 24 hours. Each sample was then extracted with distilled water. The filtrate was collected for sugar analysis via HPLC, and the cellulosic solids were dried for percent solids determination. Viscosity could not be measured in any of the samples as there was still too much heterogeneity in the sample.

Results from this experiment are displayed in FIG. 7. The total sugar content increased over the duration of the experiment to a maximum of 0.25 kg of the mixture, which equates to less than 15% saccharification conversion. During the liquefaction, the mixture remained a solid and no liquefaction was observed regardless of sugar conversion. This lack of performance is attributed to the biomass comprising large amount of lignin, which is known to deactivate enzymes due to adsorption processes. This experiment demonstrates that the use of a high-quality cellulose with a very low lignin content is desirable to obtain liquefaction using a mixing vessel described herein.

### Experiment #5

A high purity cellulose obtained from the delignification of canola biomass using a modified Caro's acid was used for this experiment. After the delignification, cellulosic solids were separated from the liquid stream using a filter press and neutralized to a pH 6-7 using NaOH. The cellulose was characterized prior to exposing it to the liquefaction step. The characterization is found in Table 8. The Kappa number was measured as TAPPI method T236. Ash content was measured as per TAPPI method T211, and the distribution of cellulose and hemicellulose content was determined as per TAPPI method T203.

TABLE 8
Characterization of the cellulosic solids obtained from
the delignification of canola biomass used Experiment #5.

| Parameter | Result |
|---|---|
| Kappa number | 1.9 |
| Lignin content (% wt.) | 0.29% |
| Cellulose content (% wt.) in Pulp | 92.7 |
| Hemicellulose content (% wt.) in Pulp | 7.3 |

The cellulosic material (high purity cellulose) was added as a “never dried” product to the mixing vessel equipped with interwoven ribbon blades. A citrate buffer concentrate was prepared and pumped into the mixing vessel. The slurry was heated up to a temperature of 50° C. and the auger in the screw reactor was rotated at 22.5 revolutions per minute, changing direction every 30 minutes. The cellulosic material with the buffer was left to distribute homogeneously before taking a t=0 sample. A cellulase enzyme cocktail was then added to the reactor at 0.10% protein per gram of the weight of cellulosic solids. The amounts of cellulosic material, buffer and enzyme were selected to obtain a dry cellulose loading of 21% wt. of the entire reaction mixture.

Samples were collected every 2 hours for a period of 24 hours. Each sample was then extracted with distilled water. The filtrate was collected for sugar analysis via HPLC, and the cellulosic solids were dried for percent solids determination. After 24 hours, the material was transferred into a CSTR and further saccharified for another 15 hours. After 15 hours, the cellulase enzyme cocktail was topped up with an additional 0.09 wt. % protein per gram of the weight of cellulosic solids. The saccharification then continued for an additional 67.5 hours. After saccharification, yeast was added at 0.4% wt. loading for fermentation under facultative aerobic conditions.

The results of Experiment #5 are shown in FIG. 8 and FIG. 9. As shown in FIG. 8, during the liquefaction stage, the cellulose liquified within 2 hours, measuring at a viscosity of approximately 10.6 Pa·s. After 24 hours, that viscosity had decreased by more than 50%, measuring at approximately 4.7 Pa·s. Cellulose content demonstrated a slow decrease over the course of the liquefaction, with the formation of sugars and glucose showing a proportional increase. FIG. 9 displays the results of the liquefaction, as well as the continued saccharification in the CSTR. The decrease in viscosity and cellulose content followed similar trends of decreasing over time. After a total reaction time of 24 hours of liquefaction, followed by an additional 82.5 hours of scarification, the yield of glucose reached 1.4 kilograms, and the yield of total sugars reached a yield of 2.2 kilograms. These values represent 41% and 64%, respectively, of the conversion of added cellulose. After 70 hours of fermentation, the ethanol converted from the total sugars produced as a result of the liquefaction and saccharification stages was equivalent to 86%. The overall conversion of the starting cellulose to ethanol, was 56%. These results demonstrate that this process is an effective approach for producing cellulosic ethanol. The first stage of liquefaction enables a much higher cellulose loading than what is commonly seen in industry. The resulting slurry can then be pumped to a typical CSTR for further saccharification and finally the final stage of fermentation has an efficient conversion of the resulting sugars to ethanol. Addition of the liquefaction stage will maximize yields process efficiency, while maintaining high-value product yields.

### Experiment #6

In this experiment, a high purity cellulose obtained from the delignification of canola biomass using a modified Caro's acid and the corresponding canola biomass were used. For the high purity cellulose, after the delignification, cellulosic solids were separated from the liquid stream using a filter press and neutralized to a pH 6-7 using NaOH. The cellulose was characterized prior to exposing it to the liquefaction step. The characterization is found in Table 9. The Kappa number was measured as TAPPI method T236. Ash content was measured as per TAPPI method T211, and the distribution of cellulose and hemicellulose content was determined as per TAPPI method T203.

TABLE 9
Characterization of the cellulosic solids obtained from the
delignification of canola biomass used in Experiment #6.

| Parameter | Result |
|---|---|
| Kappa number | 2.0 |
| Lignin content (% wt.) | 0.30% |
| Ash content (% wt.) | 13.58% |
| Pulp content (% wt., cellulose and hemicellulose) | 86.12% |
| Cellulose content (% wt.) in Pulp | 85.70% |
| Hemicellulose content (% wt.) in Pulp | 14.30% |

The canola straw was characterized to determine the content of acid-insoluble lignin (also known as Klason lignin) as well as carbohydrate composition. The carbohydrate composition gives an indication of cellulose and hemicellulose distribution within the biomass. Results of the characterization of the canola straw are shown in Table 10.

TABLE 10
Results of the characterization of
canola straw used in Experiment #6

| Parameter | Units | Result |
|---|---|---|
| Klason lignin | %, OD basis | 16.1% |
| Arabinan | %, extracted OD basis | 1.2% |
| Xylan | %, extracted OD basis | 11.4% |
| Mannan | %, extracted OD basis | 1.0% |
| Galactan | %, extracted OD basis | 1.1% |
| Glucan | %, extracted OD basis | 24.9% |

### Experimental Procedure

The cellulosic material was added as a never dried product to the mixing vessel equipped with interwoven ribbon blades. For the canola straw experiment, the straw was added to the mixing vessel followed by sulfuric acid. In both cases, the final concentration of the acid in the reaction mixture is 5% wt. and the final amount of solids in the reaction mixture is 15% wt. The slurry was heated to 70° C. and the auger in the screw reactor was rotated at 22.5 revolutions per minute, changing direction every 30 minutes. The material with the acid was left to distribute homogeneously before taking a t=0 sample.

Samples were collected every 2 hours for a period of 24 hours. Each sample was then extracted with distilled water. The filtrate was collected for sugar analysis via HPLC as well as for acidity, and the cellulosic solids were dried for percent solids determination.

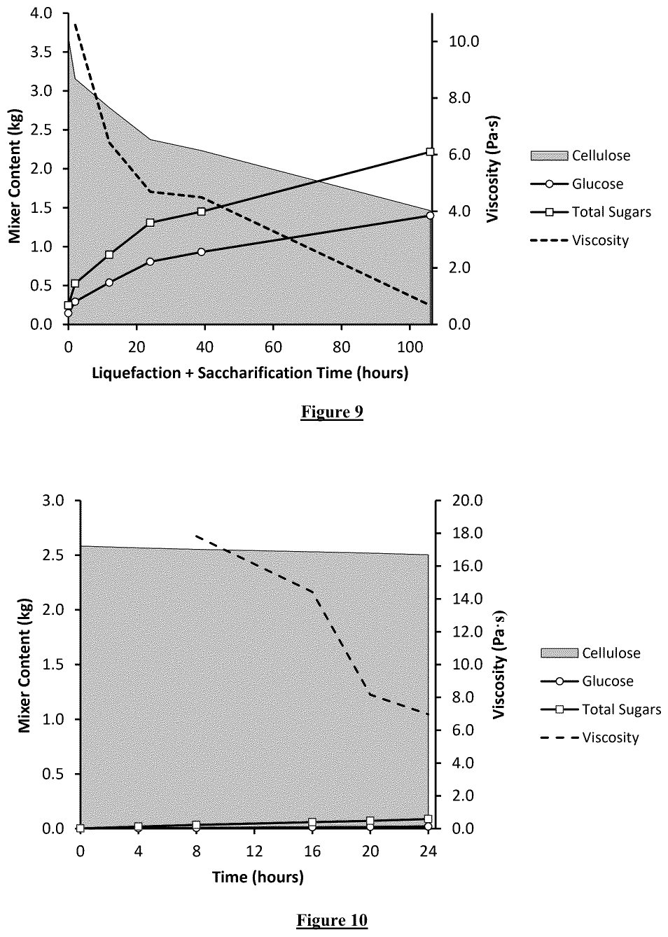

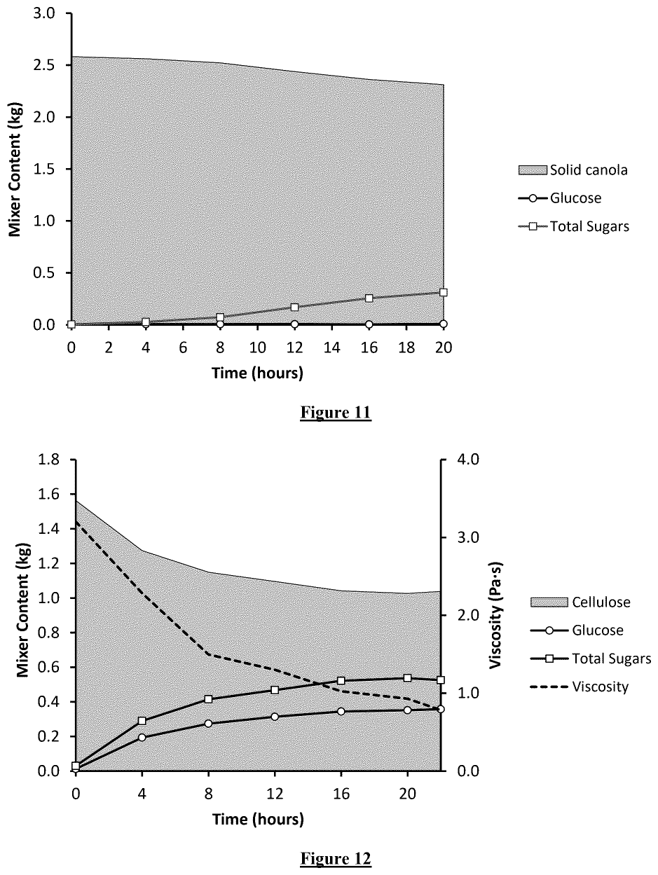

Results are shown in FIG. 10 and FIG. 11 and summarized in Table 11. During the liquefaction of the cellulose, the total sugar concentration was lower than for the canola straw. In both cases, the total sugar concentration was driven by a higher xylose/arabinose concentration, due to the preferred degradation of hemicellulose under the conditions of the tests. The higher total sugar concentration in Experiment #5 is attributed to a higher content of hemicellulose in the canola straw than in the high purity cellulose, which preferentially undergoes degradation under experimental conditions.

TABLE 11
Parameters of each of the reactions carried out in Experiment #6.

| | Experiment | Time of | Total Sugar |
|---|---|---|---|
| Feedstock | ID | mixing (h) | at end (kg) |
| Cellulose | Experiment 4-a | 24 hours | 0.17 |
| Canola straw | Experiment 4-b | 24 hours | 0.31 |

It is important to note that even though a higher total sugar concentration was observed when canola straw was used, liquefaction was not observed, and the material did not have a liquid behavior such as what was observed when a high purity cellulose was employed. Viscosity could not be measured in any of the samples when canola straw was used as the material exhibited a solid behavior with no flowability throughout the experiment. This highlights the fact that a raw lignocellulosic biomass when undergoing the process will remain non-flowable at the end of the reaction. The person skilled in the art will understand that raw lignocellulosic biomass indicates a biomass where the original components (lignin, hemicellulose and cellulose) are present in amounts similar to those found in the native plant. On the other hand, the reaction comprising the high purity cellulose liquified over the timeline of the experiment showing a final viscosity at 24 hours of 7 Pa·s. This experiment further highlights the desirability to use of a high purity cellulose to obtain optimal liquefaction when carrying out the process at high solids content materials.

### Experiment #7

In Experiment #7, the cellulosic material obtained from the delignification of a hardwood biomass was added as a never dried product to the mixing vessel equipped with interwoven ribbon blades. Sulfuric acid was added to the mixing vessel in an amount equivalent to 5% wt. of the reaction mixture, making the % solids of the reaction mixture 15% wt. The slurry was heated to 70° C. and the auger in the screw reactor was rotated at 22.5 revolutions per minute, changing direction every 30 minutes. The reaction was left 24 hours, after which the mixture had liquefied and showed a viscosity of 5.0 Pa·s. The mixture was then neutralized to pH 7.4 and transferred into a CSTR where the slurry was heated up to a temperature of 50° C. A citrate buffer concentrate was prepared and pumped into the mixing vessel. The cellulosic material with the buffer was left to distribute homogeneously before taking a t=0 sample and adding a cellulase enzyme cocktail in a concentration of 0.259% wt. enzymatic protein of dry solids. Samples were collected every 2 hours for a period of 22 hours. Each sample was then extracted with distilled water. The filtrate was collected for sugar analysis via HPLC, and the cellulosic solids were dried for percent solids determination.

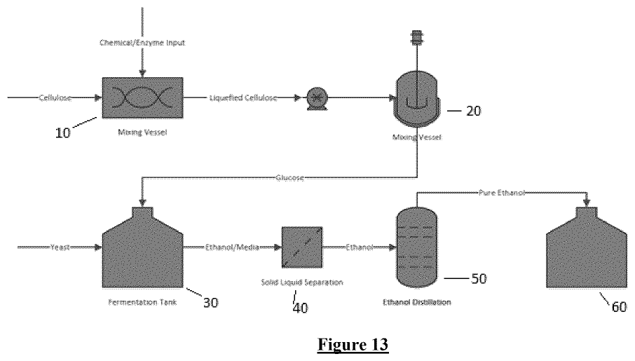

Upon neutralization and addition of the buffer, the slurry had a viscosity of 3.2 Pa's, which then decreased over time by 4-fold to a final viscosity of 0.78 Pa·s. The results from the saccharification showed great compatibility between a chemical liquefaction and a posterior enzymatic saccharification (see FIG. 12). Viscosity continued to decrease over the saccharification period, and the total sugars increased to about half of the content of the CSTR. This showcases that when following a process as described herein, a high solids mixture can be utilized to convert cellulose into various sugars that can be then used for further processing into value added chemicals.

It is believed that, by employing a high purity cellulose in conjunction with the process according a preferred embodiment of the present invention, the conversion of cellulose to depolymerized material can reach up to 80% conversion rate allowing for a flowable material. Advantageously, the proposed process according to a preferred embodiment employs a cellulose which is very low in lignin content as well as hemicellulose. By way of example, FIG. 13 displays the process in the context of the conversion to ethanol. In a first step, cellulose and either chemicals or an enzyme are mixed in a mixing vessel (10) to liquefy the cellulose. In a second step, the liquefied cellulose is removed and pumped to a second vessel (20) which uses a paddle mixer, for example, for further degradation of the liquefied cellulose to glucose. The resulting glucose is then transferred to a fermentation tank (30) where it is exposed to a fermenting organism under the appropriate conditions for fermenting it into ethanol. The resulting ethanol/media mixture undergoes solid/liquid separation in an appropriate separator (40) and the ethanol removed is then sent to a distillation unit (50) for purification of the ethanol. The purified ethanol is removed and sent to a tank (60) for further use.

In some embodiments of the present invention, the process further includes a step to separate and subsequently purify the value-added product from the rest of the fermentation stream.

According to a preferred embodiment of the present invention, the high purity cellulose is a cellulose that has not undergone any distinct bleaching steps, such as a bleaching of a pulp. Preferably, said high purity cellulose source comprises less than 15 wt. % of hemicellulose. More preferably, said high purity cellulose source comprises less than 1.5 wt. % of lignin.

The term “saccharified solution” refers to a composition comprising of mostly simple sugars such as oligo, di-, and monosaccharides (i.e., glucose, xylose, etc.). In some embodiments, the term “saccharified solution” might also be referred to compositions where some complex sugars (i.e., polysaccharides including undegraded cellulose and hemicellulose) are present.

The results obtained by the delignified biomass lends further support to the use of blended streams for generating value added products such as ethanol from a combination of sugar and starch materials and a high purity cellulose. Said high purity cellulose being defined as having a low kappa number and low hemicellulose content. Preferably, the combination of a sugar stream from each process significantly decreases the carbon intensity score of the value-added product generated when compared to the process where only the sugar or 1G sugar-containing material or starch material is employed.

The resulting liquid stream comprising a mixture of depolymerized cellulose can subsequently be used in a process to obtain a low carbon intensive fermentation stream, comprising the following steps:
exposing said mixture of depolymerized cellulose to a sugar hydrolysate obtained from the saccharification of a non-cellulose-based sugar source material, thus obtaining a combined hydrolysate stream,
optionally, exposing said combined hydrolysate stream to an organism, which converts said combined hydrolysate stream into ethanol or at least one other value-added product.

According to a preferred embodiment of the present invention, the use of a mixture of depolymerized cellulose with the another sugar hydrolysate obtained from the saccharification of a non-cellulose based organic source material results in at least 5% less carbon intensity score (in gCO₂e/MJ) for the production of the purified value added product than if said purified value added product is produced solely from the another sugar hydrolysate obtained from the saccharification of a non-cellulose based organic source material. Preferably, the reduction in carbon intensity score is more than 10%. More preferably, the reduction in carbon intensity score is more than 20%.

According to a preferred embodiment of the present invention, said non-cellulose based organic source hydrolysate is obtained from the saccharification of non-cellulose based organic source materials including, but not limited to, crops and grains (starches, cereals), such as corn, corn fiber, molasses, sugar beets, sugar cane, sweet sorghum, wheat, cassava, rye, potatoes, sorghum grain, barley, their corresponding waste materials and/or combinations thereof.

According to a preferred embodiment of the present invention, the resulting mixture (or saccharified solution) containing said cellulosic sugar hydrolysate can be combined with another sugar hydrolysate obtained from the saccharification of a non-cellulose based sugar source material in a weight ratio ranging from 99:1 to 1:99. Preferably, the ratio of lignocellulosic sugar hydrolysate to the another sugar hydrolysate obtained from the saccharification of a non-cellulose based sugar source material ranges from 80:20 to 20:80. More preferably, the ratio of lignocellulosic sugar hydrolysate to the another sugar hydrolysate obtained from the saccharification of a non-cellulose-based sugar source material ranges from 60:40 to 40:60. It is known to those skilled in the art that the ideal ratio will be that which will lead to a larger reduction in carbon intensity metrics while providing cost benefits.

According to a preferred embodiment of the present invention, the use of the cellulosic sugar hydrolysate obtained from the disclosed process can be combined with another sugar hydrolysate obtained from the saccharification of a non-cellulose based sugar source material results in at least 5% less carbon intensity score (in gCO₂e/MJ) for the production of the purified value added product than if said purified value added product is produced solely from the another sugar hydrolysate obtained from the saccharification of a non-cellulose based sugar source material. Preferably, the reduction in carbon intensity score is more than 10%. More preferably, the reduction in carbon intensity score is more than 20%.

According to a preferred embodiment of the present invention, the solid contents of the resulting mixture of depolymerized cellulose comprises less than 15% of hemicellulose. Preferably, the solid contents of the resulting mixture of depolymerized cellulose comprises less than 10% of hemicellulose. More preferably, the solid contents of the resulting mixture of depolymerized cellulose comprises less than 5% of hemicellulose. According to a preferred embodiment of the present invention, the solid contents of the resulting mixture of depolymerized cellulose may comprise hemicellulose as the fermentation organism(s) may be either able to ferment such into ethanol or be able to be engineered to ferment such into ethanol. According to a preferred embodiment, one way to ferment C5 sugars from hemicellulose is by engineering an organism that already ferments C6 sugars (cellulose) to additionally ferment C5 sugars (hemicellulose) (i.e., engineered yeasts). According to another preferred embodiment, one can employ co-fermentation using 2 organisms: one able to ferment C6 (cellulose) and one able to ferment C5 (hemicellulose). Preferably, said enzyme mixture comprises of cellulases and hemicellulases.

Those skilled in the art know that further processing of the resulting mixture can yield different at least one value-added product from the fermentation of sugar extracts or hydrolysates. The different value-added products are obtained when different fermenting organisms are employed. Examples of value-added products obtained from the fermentation of the hydrolysate obtained in the present invention include, but are not limited to, organic acids (i.e., formic acid, acetic acid), alcohols (i.e., ethanol, isopropanol, isobutanol, n-butanol, propanol), ketones (i.e., acetone), and combinations thereof. According to a preferred embodiment of the present invention, the value-added product is sorbitol. According to a preferred embodiment of the present invention, the value-added product is ethanol. According to another preferred embodiment of the present invention, the value-added product is hydroxymethylfurfural. According to another preferred embodiment of the present invention, the value-added product is selected from the group consisting: levulinic acid; chloromethylfurfural; and 2,5-furandicarboxylic acid.

While the foregoing invention has been described in some detail for purposes of clarity and understanding, it will be appreciated by those skilled in the relevant arts, once they have been made familiar with this disclosure that various changes in form and detail can be made without departing from the true scope of the invention in the appended claims.

1. A process for the liquefaction of cellulose, said process comprising:
providing a source of cellulose; wherein said source of cellulose comprises cellulose and hemicellulose, wherein said source of cellulose is non-flowable;
adding said source of cellulose to a mixing vessel; wherein said mixing vessel comprises a mixing apparatus selected from the group consisting of: a horizontal single helicoid auger flight; a horizontal double helicoid auger flight; a vertical single helicoid auger flight; a vertical double helicoid auger flight; a horizontal single ribbon auger flight; a horizontal double ribbon auger flight; a vertical single ribbon auger flight; a vertical double ribbon auger flight; a horizontal single sectional auger flight: a horizontal double sectional auger flight; and a vertical single sectional auger flight; a vertical double sectional auger flight;
adding a solution to said mixing vessel containing said source of cellulose to yield a reaction mixture wherein said source of cellulose is present in a concentration of solids ranging from 5% to 50% of the total mass of said reaction mixture;
mixing said reaction mixture in said mixing vessel until said reaction mixture has undergone liquefaction to yield a resulting mixture comprising oligomers and monomers from the degradation of cellulose and hemicellulose and wherein said resulting mixture has a resulting viscosity of no more than 15 Pa's; and
optionally, separating said resulting mixture into a liquid stream used for subsequent processing and a solid stream that is introduced back into the mixing vessel for further processing.

2. The process according to claim 1 wherein said source of cellulose has a lignin content of less than 1% of the total weight of said cellulose and a hemicellulose content of less than 15% of the total weight of said cellulose.

3. The process according to claim 1 wherein said solution comprises an acid selected from the group consisting of: sulfuric acid; hydrochloric acid; phosphoric acid; nitric acid; and a combination thereof.

4. The process according to claim 1 wherein said acid is in a concentration of up to 50 wt. % of said reaction mixture.

5. The process according to claim 1 wherein said acid is in a concentration of up to 10% wt. of said reaction mixture.

6. The process according to claim 1 wherein said acid is in a concentration of up to 5% wt. of said reaction mixture.

7. The process according to claim 1 wherein said reaction mixture is heated to a temperature of up to 150° C. during said mixing step.

8. The process according to claim 1 wherein said reaction mixture is heated to a temperature of up to 80° C. during said mixing step.

9. A process for the liquefaction of cellulose, said process comprising:
providing a source of cellulose; wherein said source of cellulose comprises cellulose and hemicellulose, wherein said source of cellulose is non-flowable;
adding said source of cellulose to a mixing vessel; wherein said mixing vessel comprises a mixing apparatus selected from the group consisting of: a horizontal single helicoid auger flight; a horizontal double helicoid auger flight; a vertical single helicoid auger flight; a vertical double helicoid auger flight; a horizontal single ribbon auger flight; a horizontal double ribbon auger flight; a vertical single ribbon auger flight; a vertical double ribbon auger flight; a horizontal single sectional auger flight: a horizontal double sectional auger flight; and a vertical single sectional auger flight; a vertical double sectional auger flight;
adding a buffer solution to said mixing vessel containing said source of cellulose to yield a reaction mixture where said source of cellulose is present in a concentration of solids ranging from 5% to 50% of the total mass of said reaction mixture;
adding an enzyme mixture to said reaction mixture;
mixing said reaction mixture in said mixing vessel until said reaction mixture has undergone liquefaction to yield a resulting mixture comprising oligomers and monomers from the degradation of cellulose and hemicellulose and wherein said resulting mixture has a resulting viscosity of no more than 15 Pas; and
optionally, separating said resulting mixture into a liquid stream used for subsequent processing and a solid stream that is introduced back into the mixing vessel for further processing.

10. The process according to claim 9 wherein said source of cellulose has a lignin content of less than 1% of the total weight of said cellulose and a hemicellulose content of less than 15% of the total weight of said cellulose;

11. The process according to claim 1 wherein said resulting mixture has a viscosity of no more than 10 Pa·s.

12. The process according to claim 9 wherein said enzyme mixture is added in an amount of approximately 0.01 to 1 wt % protein per gram of said source of cellulose.

13. The process according to claim 9 wherein said enzyme mixture in an amount of approximately 0.02 to 0.5 wt % protein per gram of said source of cellulose.

14. The process according to claim 9 wherein said reaction mixture is heated to a temperature of up to 70° C. during said mixing step.

15. The process according to claim 9 wherein said enzyme mixture comprises at least one cellulase.

16. The process according to claim 9, wherein said enzyme mixture further comprises at least one hemicellulase.

17. The process according to claim 9 wherein said buffer solution has a pH ranging from 3.0 to 8.0.

18. A process for the liquefaction of cellulose, said process comprising:
providing a source of cellulose, wherein said source of cellulose has a lignin content of less than 1% of the total weight of said cellulose and a hemicellulose content of less than 15% of the total weight of said cellulose, wherein said source of cellulose is non-flowable;
adding said source of cellulose to a mixing vessel;
adding a solution to said mixing vessel containing said source of cellulose to yield a reaction mixture wherein said source of cellulose is present in a concentration of solids ranging from 5% to 50% of the total mass of said reaction mixture;
mixing said reaction mixture in said mixing vessel until said reaction mixture has undergone liquefaction to yield a resulting mixture comprising oligomers and monomers from the degradation of cellulose and hemicellulose and wherein said resulting mixture has a viscosity of no more than 15 Pa·s; and
optionally, separating said resulting mixture into a liquid stream used for subsequent processing and a solid stream that is introduced back into the mixing vessel for further processing.

19. A process for the liquefaction of cellulose, said process comprising:
providing a source of cellulose, wherein said source of cellulose has a lignin content of less than 1% of the total weight of said cellulose and a hemicellulose content of less than 15% of the total weight of said cellulose.
adding said source of cellulose to a mixing vessel;
adding a buffer solution to said mixing vessel containing said source of cellulose to yield a reaction mixture where said source of cellulose is present in a concentration of solids ranging from 5% to 50% of the total mass of said reaction mixture;
adding an enzyme mixture to said reaction mixture;
mixing said reaction mixture in said mixing vessel until said reaction mixture has undergone liquefaction to yield a resulting mixture comprising oligomers and monomers from the degradation of cellulose and hemicellulose and wherein said resulting mixture has a resulting viscosity of no more than 15 Pa·s.
optionally, separating said resulting mixture into a liquid stream used for subsequent processing and a solid stream that is introduced back into the mixing vessel for further processing.

20. The process according to claim 1 wherein said reaction mixture is non-flowable at the start of the mixing step.
